(19) United States
(12) Patent Application Publication
(10) Pub. No.: US 2005/0059858 A1
(43) Pub. Date: Mar. 17, 2005

(54) Endoscope magnetic rocker switch

(76) Inventors: Martin A. Frith, Goleta, CA (US); Craig J. Speier, Santa Barbara, CA (US)

Correspondence Address: GENE WARZECHA;LINVATEC CORPORATION, 11311 CONCEPT BOULEVARD, LARGO, FL, 33773, US

(21) Appl. No.: 10/663,261

(22) Filed: Sep. 16, 2003

(51) Int. Cl. A61B 1/00

(52) U.S. Cl. 600/118; 200/553

(57) ABSTRACT

A rocker switch on an endoscopic camera allows multiple functions from a single Hall effect sensor. The switch features a neutral position between a rocked forward and a rocked back position. When the front of the switch is depressed a dome spring under the front is flattened and the magnet located in the rear is moved away from the sensor. Releasing the switch returns it to the neutral position. Depressing the rear of the switch brings the magnet closer to the Hall effect sensor while depressing another dome spring under the rear of the switch. Again, releasing the switch returns it to the neutral position.

### FIELD OF THE INVENTION

The field of this invention is switches and more particularly Hall effect switches used on endoscopic devices for actuating a variety of functions.

### BACKGROUND OF THE INVENTION

Endoscopes have become widely utilized in surgery for viewing body cavities and organs to permit performance of diagnostic and surgical procedures internally without the need for invasive surgical procedures. An endoscope is typically inserted through a small incision or portal providing access to the body cavity. A lens at a distal end of the endoscope is positioned to receive light reflected from a site to be observed, and images of the site can be viewed remotely to conduct diagnostic examinations and to perform closed, or endoscopic surgery. As used herein, the term endoscope refers generically to viewing devices for remotely observing otherwise inaccessible body cavities with minimal trauma and intrusion, including but not limited to arthroscopes, colonoscopes, bronchoscopes, hysteroscopes, cystoscopes, sigmoidoscopes, laparoscopes and ureterscopes, etc.

Endoscopes are sometimes supplied with an eyepiece at the proximal end thereof, and relay lenses in the endoscope typically produce an image for direct viewing through the eyepiece. However, adaptation of video camera technology to endoscopy imaging has enabled the output image of an endoscope to be viewed on a video monitor. Specifically, a video camera is electronically coupled to the video monitor and optically and mechanically coupled with the proximal end of the endoscope. Indirect or video monitor viewing of endoscopic images provides numerous benefits over direct viewing through an eyepiece, including: protection of a direct viewer's vision from high intensity illumination passed through the endoscope and reflecting off bodily tissue; enhancement of operator comfort and freedom of movement; increased endoscope utility and efficiency; reduction in the time required to conduct many endoscopic procedures; simultaneous viewing of endoscopic images by more than one person; and recordation and real time transmission of images of surgical procedures.

Endoscopes allow the surgeon to view the surgical site during procedures through small incisions. Typically, the endoscope is used in combination with a video camera and a light source to enable the surgeon to view the output image on a video monitor. External controls are typically provided on the camera to be operated by the surgeon. The surgeon can operate such controls to take a picture, control a video recorder, or to operate the camera to change its operating parameters. Hall effect sensors have been used in cameras in conjunction with switches to perform such operations as described above. Typically, the sensor is embedded in the inner camera housing which is distinct from an outer cover sleeve that contains the switch. The switch includes a magnet that is selectively brought in range of the sensor to change its output in proportion to the magnetic field strength. This change in sensor output can subsequently trigger the functions described above in furtherance of use of the endoscope and camera system. The operation of Hall effect devices depends on close proximity between the magnet and the Hall sensor. Because the Hall sensor is embedded in the inner camera housing with no mechanical or electrical connection with the external switch assembly, the camera can be autoclaved and effectively sterilized without any damage to the sensor or other internal sensitive electrical and optical components. The switch assembly is designed to facilitate sterilization and utilizes materials that can withstand the rigorous environment of repeated autoclave cycles.

FIG. 1 illustrates an example of a known Hall effect switch used in endoscopes. The drawing is a section view through the switch and the sensor. The sensor 10 is mounted in the inner camera housing 12 close to the surface 14. Housing 12 has a recess 16 in which is deposited a switch assembly 18. The assembly 18 comprises a receptacle 20 with a dome spring 22 residing near its lower end 24. A magnet 26 sits on top of an actuator 28. Actuator 28 has a tab 30 that engages the dome spring 22 when the surgeon pushes down on the button housing 32 that is retained in receptacle 20 by retaining ring 34 at thread 36. In the switch of FIG. 1 the switch is limited to enabling a single function. The use of a large mass of rubber for the button housing 32 dampens any audible sound made by the switch, when actuated, and severely reduces or eliminates the tactile feedback from the dome spring 22. Another disadvantage is that the magnet 26 does not have completely predictable movements in response to pressure on the button housing 32. Accordingly, its function is not always assured. Additionally, the switch shown in FIG. 1 is relatively expensive to make.

Rocker switches and other types of switches that use magnets are shown in U.S. Pat. Nos.: 5,523,730; 5,666,096 and 5,867,082 and in U.S. application 2003/0067371 A1. What is needed in an endoscopic device is a switch that can handle multiple functions, using a single Hall sensor, while retaining the ability to provide tactile and audible feedback and ease of manufacturing. It needs to reliably position the magnet for consistent switch operation and the overall assembly needs to be durable for the intended service. These and other advantages will be more apparent to those skilled in the art from a review of the preferred embodiment and the claims that appear below.

### SUMMARY OF THE INVENTION

A rocker switch on an endoscopic camera allows multiple functions from a single Hall effect sensor. The switch features a neutral position between a rocked forward and a rocked back position. When the front of the switch is depressed a dome spring under the front is flattened and the magnet located in the rear is moved away from the sensor. Releasing the switch returns it to the neutral position. Depressing the rear of the switch brings the magnet closer to the Hall effect sensor while depressing another dome spring under the rear of the switch. Again, releasing the switch returns it to the neutral position.

### BRIEF DESCRIPTION OF THE DRAWINGS

FIG. 1 is a section view of a prior art switch for an endoscopic camera.

FIG. 2 is a section view of a single Hall sensor embodiment of the switch, shown in the neutral position.

FIG. 3 is the switch of FIG. 2 shown in the rear-depressed position.

FIG. 4 is the switch of FIG. 2 shown in the front depressed position.

FIG. 5 is a see through perspective view of an alternative design for the switch using two sensors and two magnets.

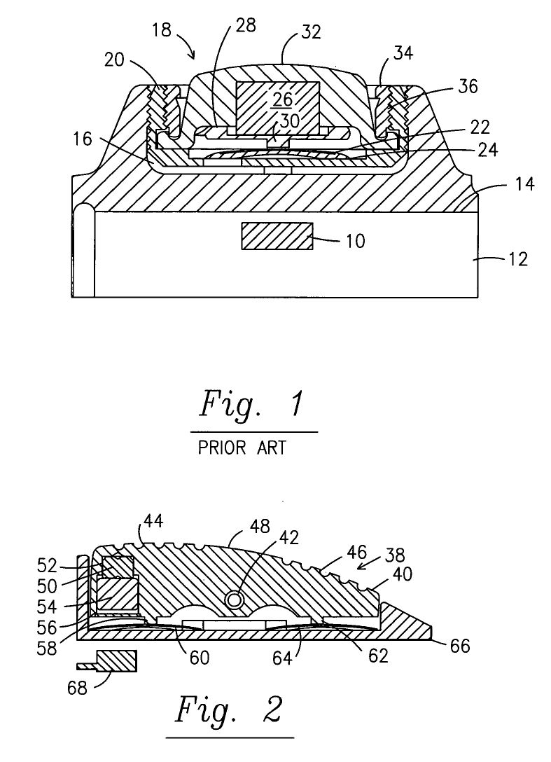

### DETAILED DESCRIPTION OF THE PREFERRED EMBODIMENT

FIG. 2 illustrates the switch 38 in the neutral position. Switch 38 has a rocker 40 pivoted at 42 with rear undulations 44 and front undulations 46 on top surface 48. A magnetic pin 50 is secured in a counter-bore 52. The magnet 54 is disposed in cavity 56 and is secured to the magnetic pin 50. Tab 58 extends down from rocker 40 toward dome spring 60. Tab 62 extends from rocker 40 toward dome spring 64 near the front of switch 38. In the neutral position of FIG. 2, both dome springs 60 and 64 are slightly, equally compressed. The switch 38 is disposed in an outer housing 66 that slides over the camera body (not shown except for the Hall sensor 68 that is preferably embedded in the camera body or the housing).

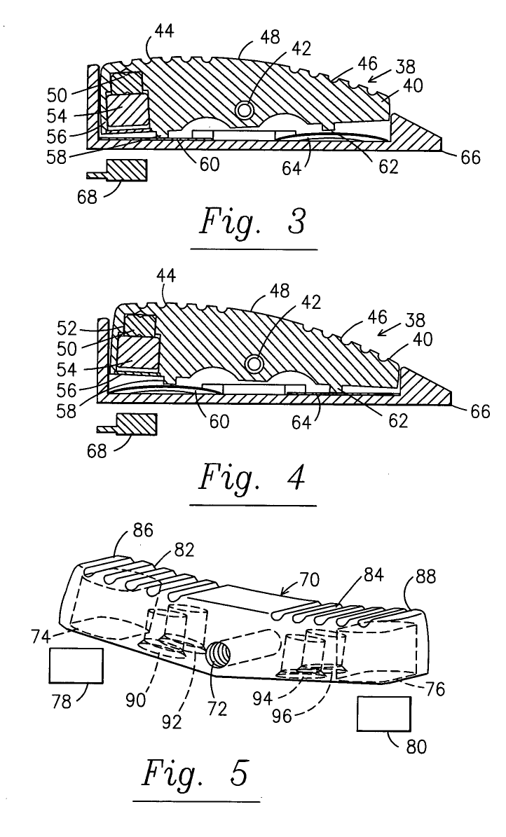

In operation, the surgeon depresses the rear of the switch 38, as shown in FIG. 3. This forces a pivoting motion about pivot 42 as the dome spring 60 is flattened or depressed and the magnet 54 arcs closer to the Hall sensor 68 to trigger a first desired function of the switch 38. When the switch is released by the surgeon from the FIG. 3 position, it is returned to the neutral position of FIG. 2 by the action of dome spring 60 releasing the stored force in it resulting from it being compressed into the FIG. 2 position.

As shown in FIG. 4, a push on the front of switch 38 flattens or depresses dome spring 64 and pivots the magnet 54 away from Hall sensor 68. In this position another function of the endoscopic camera can be accomplished. Again, releasing the switch when it is in the FIG. 4 position simply brings it back to the neutral position of FIG. 2.

Those skilled in the art will readily see the advantages of this embodiment. The switch 38 can be set in the FIG. 3 or FIG. 4 position to accomplish different functions on the camera. These discrete functions are accomplished with a single sensor. The placement of the magnet 54 with respect to the sensor 68 is assured and is repeatable. The switch 38 is simple to construct and allows for reliable long-term operation. The dome springs 60 and 64 allow for audible and tactile feedback. The switch automatically, returns to a neutral position when released.

Referring now to FIG. 5, switch 70 pivots at pivot 72. It features cavities 74 and 76 on opposite ends that are respectively in alignment with Hall sensors 78 and 80. Magnets, not shown, are inserted into cavities 74 and 76. Bores 90 and 92 are disposed on one side of pivot 72 while bores 94 and 96 are disposed on the other side of pivot 72. Compression springs, not shown, are inserted into these bores to achieve a neutral position of the switch 70 where both the magnets are equally spaced from their respective Hall sensors 78 and 80. Undulating surfaces 82 and 84 respectively on the rear 86 and the front 88 provide for an improved grip. In this embodiment two or more discrete functions are possible using at least a pair of magnets opposite a pair of Hall sensors 78 and 80. All the other stated benefits of the embodiment of FIGS. 2-4 are also achieved in the FIG. 5 embodiment. The primary difference is that additional magnets and sensors are used. The springs in the bores can be of a variety of types but coiled springs are preferred. As before, the switch 70 is installed in an outer sleeve that fits over the camera body that houses the Hall sensors. This allows the camera to be sterilized without affecting the sensors and the associated circuits that are sealed within the camera body. Still another embodiment includes the Hall sensors mounted in the switch assembly and the magnets mounted in the inner camera housing. Similar to the previously described embodiments, the operation of the switch would either increase or decrease the proximity of the Hall sensor and magnet, providing the same functionality.

The above description is illustrative of the preferred embodiment and many modifications may be made by those skilled in the art without departing from the invention whose scope is to be determined from the literal and equivalent scope of the claims below.

1. An endoscopic camera comprising:
a body;
a rocker switch;
at least one magnet;
at least one Hall effect sensor;
said rocker switch altering the distance between said magnet and said Hall effect sensor for controlling a plurality of operations of the endoscopic camera.

2. The endoscopic camera of claim 1 wherein:
said rocker switch facilitates a first function of the endoscopic camera when the distance between said Hall effect sensor and said magnet is decreased and a second function of the endoscopic camera when said distance between said Hall effect sensor and said magnet is increased.

3. The endoscopic camera of claim 2 further comprising:
a neutral position between said minimum and maximum spacing of said Hall effect sensor and said magnet.

4. The endoscopic camera of claim 3 wherein:
said rocker switch comprises a pivot on said body located between a front and a rear end of said rocker, said magnet disposed adjacent one of said ends of said rocker switch.

5. The endoscopic camera of claim 3 further comprising:
at least one spring to bias said rocker switch toward said neutral position.

6. The endoscopic camera of claim 5 wherein:
said at least one spring comprises a plurality of springs to bias said rocker switch to said neutral position from said decreased and increased spacing of said Hall effect sensor and said magnet.

7. The endoscopic camera of claim 5 wherein:
at least one said spring is disposed on each side of a pivot connection on said rocker switch.

8. The endoscopic camera of claim 7 wherein:
said springs comprise dome springs.

9. The endoscopic camera of claim 1 wherein:
said Hall effect sensor is embedded in said body and said magnet is mounted on said rocker switch with said rocker switch mounted to a surrounding housing around said body.

10. The endoscopic camera of claim 9 wherein:
said surrounding housing is removably mounted to said body.

11. The endoscopic camera of claim 10 wherein:
said rocker switch is mounted on a pivot to said surrounding housing and said pivot is positioned between a front and rear end of said rocker switch, said magnet is mounted near one of said ends of said rocker switch in alignment with said Hall effect sensor.

12. The endoscopic camera of claim 11 wherein:
said rocker switch comprises a neutral position between said Hall effect sensor and said magnet, wherein said increased and decreased distance positions control different functions of the endoscopic camera.

13. The endoscopic camera of claim 12 wherein:
said rocker switch is biased toward said neutral position from either said increased or decreased distance positions.

14. The endoscopic camera of claim 1 wherein:
said at least one Hall effect sensor comprises a plurality of Hall effect sensors;
said at least one magnet comprises a plurality of magnets; and
wherein rocking said rocker switch brings different pairs of Hall effect sensors and magnets closer together.

15. The endoscopic camera of claim 14 wherein:
said rocker switch further comprises a neutral position where the distance between pairs of Hall effect sensors and magnets are substantially equal.

16. The endoscopic camera of claim 15 wherein:
said rocker switch is biased toward said neutral position.

17. The endoscopic camera of claim 14 wherein:
placement of a different pairs of magnets and Hall sensors in closer proximity controls an independent function of the endoscope.

18. The endoscopic camera of claim 14 wherein:
said rocker switch is pivotally mounted on a pivot located between a front and rear end thereof and said magnets are located in said rocker switch near said front and rear ends with each said Hall effect sensor located in alignment with a corresponding said magnet.

19. The endoscopic camera of claim 18 wherein:
said rocker switch is mounted to a housing surrounding and removably mounted to said body and said Hall sensors are mounted within said body.

20. The endoscopic camera of claim 19 wherein:
said rocker switch further comprises a neutral position where the distance between pairs of Hall effect sensors and magnets are substantially equal; and
said rocker switch is biased toward said neutral position.
